(19) United States
(12) Patent Application Publication
(10) Pub. No.: US 2005/0267457 A1
(43) Pub. Date: Dec. 1, 2005

(54) Tissue ablation device using a lens to three dimensionally focus electromagnetic energy

(76) Inventor: James Anthony Hruschka, Albuquerque, NM (US)

Correspondence Address: James Anthony Hruschka, 2132 Central SE # 186, Albuquerque, NM, 87106, US

(21) Appl. No.: 11/136,212

(22) Filed: May 24, 2005

Related U.S. Application Data
(60) Provisional application No. 60/574,528, filed on May 25, 2004.

(51) Int. Cl. A61B 18/18

(52) U.S. Cl. 606/33; 607/100; 607/101

(57) ABSTRACT

This invention provides a device that uses a lens to focus electromagnetic energy to a singular three dimensionally focused point. The invention has three-dimensional target positioning capability. This invention can be used in tissue ablation, tissue thermography, tissue heating, and tissue hyperthermia. Possible specific treatment applications include, but are not limited to: cancer ablation, scar tissue ablation, epithelial tissue ablation, infected tissue ablation, herniated disk removal, tissue growth stimulation. The main advantage of this device is that it maximizes the effect on the target tissue and greatly reduces the effect of the surrounding tissue to the point of there being close to no side effects from radiation therapy.

This application claims priority from U.S. Provisional Application No. 60/574,528, filed May 25, 2004.

### BACKGROUND OF THE INVENTION

The current invention relates to the use of a lens to focus electromagnetic energy toward a target at a specific three-dimensional point in space. Specifically the present invention is for use in medical application affecting diseased or damaged tissue.

Excision or destruction of diseased or damaged tissue in a living being has long been known in the art. Common means are surgical incision, ultrasound, and the use of electromagnetic radiation, including: X-ray, microwave, radio frequency, infra-red, visible spectra and ultra-violet radiation. All of these have the disadvantage of damaging or destroying healthy tissue as well as the target tissue. Electromagnetic systems are described in U.S. Pat. No. 4,672,980, “System and method for creating hyperthermia in tissue” U.S. Pat. No. 4,934,365, “Non-invasive hyperthermia method and apparatus”; U.S. Pat. No. 5,251,645, “Adaptive nulling hyperthermia array”; U.S. Pat. No. 6,400,980, “System and method for treating select tissue in a living being”; U.S. Pat. No. 6,477,426, “System and method for heating the prostate gland to treat and prevent the growth and spread of prostate tumors”. Generally these inventions emit electromagnetic energy toward a specific target destroying both target and non-target tissue. U.S. Pat. No. 5,097,844, “Hyperthermia apparatus having three-dimensional focusing” refers to the use of multiple energy generators to have the effect of three-dimensional focusing.

As energy passes through tissue, some of the energy is absorbed, creating an increase in temperature of the tissue. The increase in temperature correlates to the amount of energy absorbed. The more energy that passes through a given volume of tissue, the greater amount of energy absorbed, and the greater increase in tissue temperature. When the tissue reaches a certain temperature, the tissue dies.

A major problem with the current technology is the damage or destruction of healthy tissue. Current devices direct electromagnetic energy such that upon entering the surface tissue, some of the electromagnetic energy is absorbed by the tissues closest to the penetration point, with the non-absorbed energy passing through to the next layer of tissue. The next layer of tissue absorbs some of the energy, again with the non-absorbed energy passing through to the next layer of tissue. This cycle is repeated until either all of the energy is absorbed by the person's body or the remaining energy completely passes through the person's body and is absorbed by a material beyond the person's body. All tissue layers that absorb enough energy are destroyed whether healthy or diseased. In this method, the tissue destruction is often caused by the absorbed energy causing a heating effect, which denatures the proteins, lipids, RNA, and/or DNA. Tissue destruction can also be caused by non-thermal radiation damage (such as X-rays) which has similar destructive effects. Both thermal and non-thermal methods have the possible undesired side effect of mutating healthy tissue into cancerous tissue. Both methods may also have the side effect of destroying healthy tissue as well as the target tissue. If done properly, the target tissue is one of the absorbing layers of tissue. However, the surface layer of tissue is hit with the most energy, with each layer underneath being hit by less energy. With most electromagnetic medical tissue ablation devices, intervening tissue between the source of the electromagnetic energy and the target tissue is destroyed or mutated. This problem can be solved with the use of a lens to maximize the effect of the electromagnetic energy on the target and minimize the effect of the electromagnetic energy on healthy non-target tissue.

In U.S. Pat. Nos. 5,059,192, Oct. 22, 1991; 4,388,924, Jun. 21, 1983; 5,226,907, Jul. 13, 1993; reliance is placed on selective photothermolysis; that is the selective absorption of the incident laser radiation by the melanin in the follicle to cause localized heating.

In U.S. Pat. No. 5,632,741, May 27, 1997, and related WO98/25673, Jun. 18, 1998, both Zavislan et al, a lens is used, but only for surface epilation by a visible spectra laser, and is not designed for sub-dermal ablation.

U.S. Pat. No. 5,320,617, Jun. 14, 1994, uses a lens for the invasive insertion of fiberoptic laseroscope into the urethra, and is only usable for destroying prostate tissue or other tissue that can be reached via insertion of a fiberoptic tube. The energy emitted from the fiberoptic tube has minimal penetration depth.

There are several methods described in the art for changing the direction of electromagnetic energy: U.S. Pat. Nos. 5,170,167, “Reflector for electromagnetic energy; 6,424,318, “Method and arrangement pertaining to microwave lenses”; 6,562,448, “Low density dielectric having low microwave loss”; 6,660,193, “Method of manufacturing a lens for microwave frequencies”. A method for creating microwave lenses has been created in U.S. Pat. No. 6,660,193. Other lenses exist as well. It is possible that the use of a lens suitable for focusing electromagnetic energy for subsurface medical application has been overlooked in the past because lenses for use with electromagnetic energy outside the visible spectra are fairly new inventions. However, visible spectra medical devices sometimes incorporate a lens, but visible spectra electromagnetic energy thus produced has very limited penetration depth.

The current invention provides a medical device that uses a lens to focus electromagnetic energy to a singular three-dimensionally focused point. Using a lens creates the effect of the focal point having maximal energy per volume, while there is an ever decreasing amount of energy per volume the further away from the focal point the non-target tissue is.

The current invention has three-dimensional target positioning capability. This invention can be used in tissue ablation, tissue thermography, tissue heating, and tissue hyperthermia. Possible specific treatment applications include, but are not limited to: cancer ablation, scar tissue ablation, epithelial tissue ablation, infected tissue ablation, herniated disk removal, tissue growth stimulation. Infra-red, microwave and radiofrequency have wavelengths of greater than 7×10⁻⁷ (m), and can be used for thermal ablation. X-rays and Gamma-rays have wavelengths of less than 1×10⁻⁸ (m), and can be used for radiation treatment. Visible spectra and UV spectra have wavelengths between 7×10⁻⁷ (m) and 1×10⁻⁸ (m) and can be used for limited depth treatment. Various wavelengths can be used for varying effects.

Although other devices have been created for tissue ablation, this device has the advantage of focusing the electromagnetic energy to a three dimensionally focused point by using a lens. The lens creates maximum effect at a singular target point, and minimizes the effect on surrounding tissue. The lens takes diffuse energy and focuses it to a single point. This results in diffuse energy at the non-target tissue and concentrated energy at the target focal point. An essential difference between the present invention and the invention of U.S. Pat. No. 5,097,844 is that the present invention uses a lens to focus the electromagnetic energy to the target at a particular three-dimensional point, while U.S. Pat. No. 5,097,844 uses multiple electromagnetic energy generators to focus electromagnetic energy to the target at a particular three-dimensional point.

My device approaches the ideal of affecting only the target tissue, while minimizing the effect on the non-target tissue. Below a certain threshold of energy absorption, tissue is not damaged or can sufficiently repair itself with no clinically discernable side-effects. Using a lens maximizes the effect of the energy on the target tissue while exponentially minimizing the effect on the non-target tissue. While the actual effect cannot be accurately calculated because of changing variables in the tissue, an estimate of the minimizing effect on the non-target tissue can be calculated. Some of the variables include: molecular composition of the tissue absorbing the energy, rate of fluid flow through the tissue, movement of tissue, scattering of the energy, wavelength of energy used. A lens effectively makes a double cone of energy, with the lens being the base and the focal point (target) being the tip of the first cone. The energy continues through the target, spreading out into a second cone shape that continues until it is absorbed. In an example using energy wavelengths that result in heating the target to the point of tissue ablation, the current invention could heat a target of zero to one millimeters from the focal point (target) twenty degrees Celsius while the tissue layer one to two millimeters outside the focal point would be only be heated approximately three degrees Celsius, and the tissue layers two or more millimeters away from the focal point would be heated less than two degrees Celsius. This effectively means that only the target tissue would be heated to the point of destruction, while the surrounding tissue would have minimal or no side effects.

Other problems with using electromagnetic energy to affect sub-surface tissue are that the target cannot be seen, normal cardiovascular and respiratory activity constantly moves the target, and the electromagnetic energy used for treatment is rarely visible. A variety of targeting devices can be used to solve these problems: magnetic resonance imaging (MRI), magnetic resonance spectroscopy imaging (MRSi), X-rays, CAT scans, temperature probes, ultrasound imaging, infrared, UV/visible light fluorescence, Raman spectroscopy or microwave imaging etc. For example, an MRI device could be used for targeting; a computer controlled movement device could move the electromagnetic treatment device to an optimal position based on feedback from the MRI; temperature probes or the MRI could give feedback to the computer as to when to turn off the treatment device. When the target moves 3 mm to the left, the MRI can sense this, the computer (control box) can use this information to move the electromagnetic device 3 mm to the left to compensate, keeping the focal point of the lens on the target at all times. A description of the use of the lens and targeting device is in the detailed description and depicted in the figures.

### SUMMARY OF THE INVENTION

The object of the current invention to provide a device for producing 3-dimensional (3-D) focusing at a target within a subject's body. Another object is to minimize the effect of the electromagnetic energy on the non-target tissue. Briefly stated the invention is composed of an electromagnetic energy device coupled with a lens to provide both 3-dimensional (3-D) focusing toward the target tissue and a minimal effect on the surrounding tissue. This invention relates to systems for using focused energy to heat, ablate or otherwise affect tissue in a living body. The primary focusing device is a lens used for focusing electromagnetic energy. The invention employs a computerized imaging system (such as CAT scan, MRI, ultrasound imaging, infrared, X-ray, UV/visible light fluorescence, Raman spectroscopy or microwave imaging) to locate the area of tissue to be ablated and to very precisely focus the energy used so that tissue surrounding the selected areas is not harmed. A method for keeping the energy on a moving target is outlined. A computer can coordinate temperature readings, pulsing rates, diagnostic data, data from other diagnostic devices and imaging techniques.

### BRIEF DESCRIPTION OF THE DRAWINGS

FIG. 1 is a schematic view in block form of the three-dimensional (3-D) energy generating device coupled with a control box and a display;

FIG. 2 is a cut away view of FIG. 1 showing the energy generator, focusing lens and optional lens protector;

FIG. 3 is a conceptual view of the energy generating device showing emitted energy focused through lens, and the energy passing through the target;

FIG. 3A is a conceptual view of the focusing effect of the lens showing minimal effect on the subject's surface and maximal effect at the target area;

FIG. 4 is a conceptual view of the prior art devices that do not use a lens showing maximal effect at the subject's surface and less effect at the desired target;

FIG. 5 is a schematic view of two energy generating devices focused on one target area;

FIG. 6 is a schematic view of the use of the energy generating device combined with a target sensor;

FIG. 7 is a schematic view of the energy emitting device attached to a rotatably connected positioning device.

### DETAILED DESCRIPTION OF THE INVENTION

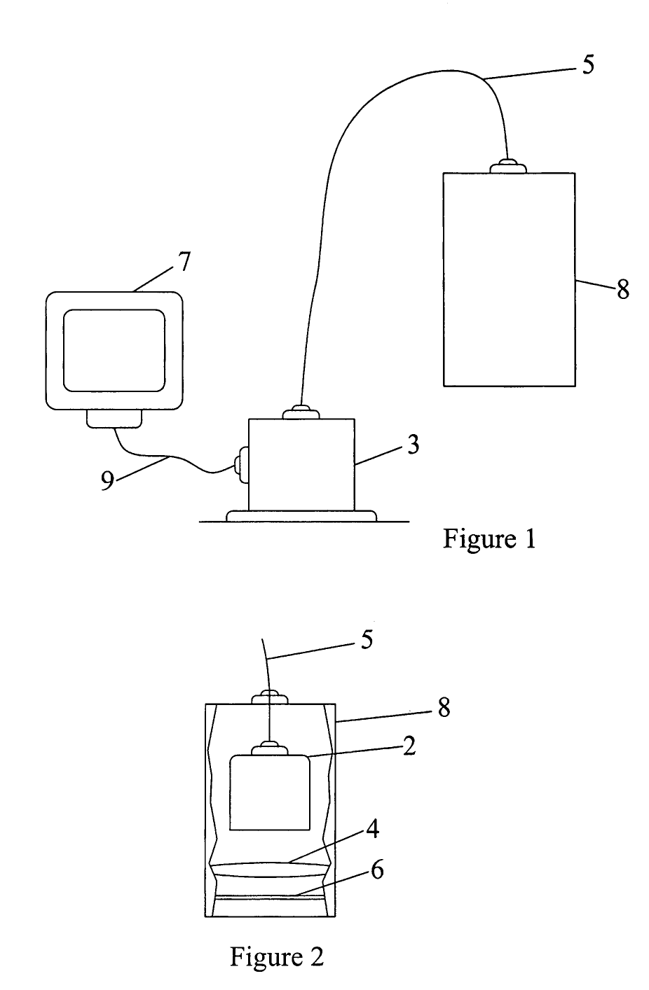

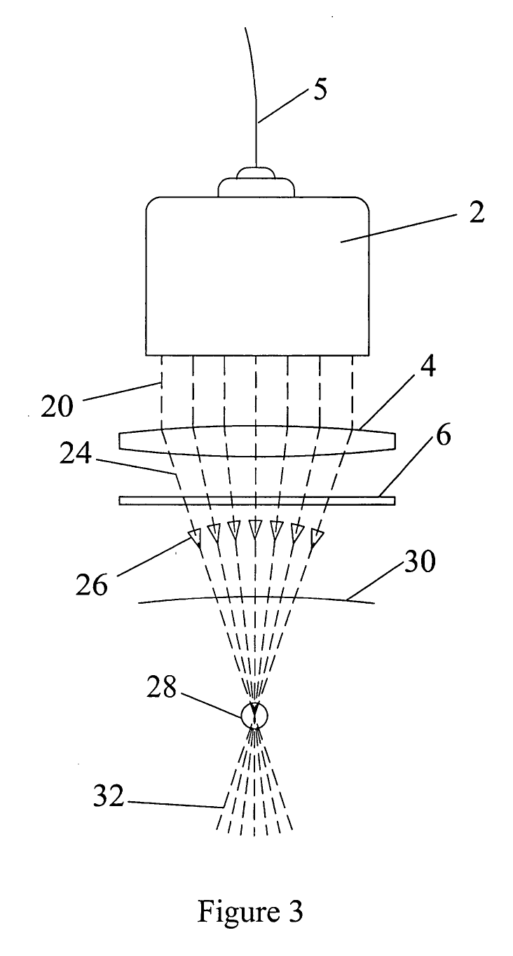

The three-dimensional (3-D) focusing device (FIG. 1 and cut-away view FIG. 2) includes electromagnetic energy generator 2 connected to control box 3 with communication link 5. Control box 3 is connected to display 7 with second communication link 9. All communication links described herein are used for transmitting information to and/or from each component. For example, the information transmitting could be data that controls the rate and output of generator 2 or it could be displayed on display 7. Optionally, communication links 5 and 9 may be replaced with a non-wired communication system, such as: infra-red, radiofrequency, etc.; it is important to have a communication system that neither interferes with nor is affected by the electromagnetic generator or other components. Control box 3 can be a computer or any other input/output device as is know in the art sufficient for operation of the invention. All communication links above and below can also include a power source or power cord connected to a power source. The electromagnetic energy coming from generator 2 is focused with focusing lens 4. After passing through lens 4, electromagnetic energy passes through optional lens protector 6. Protective casing 8 of sufficient size to enclose generator 2, lens 4, lens protector 6, and any other parts required to make the device functional. Lens protector 6 is of a material that allows electromagnetic energy to pass through it with minimal distortion. To optimize the effect of the device, the size and shape of lens 4 can be modified to change the angle of refraction.

The electromagnetic energy generated can be of a variety of predetermined or variable wavelengths. The electromagnetic energy can be pulsed, creating a photo acoustic effect for less absorption by the intervening tissue. Various intensities of electromagnetic energy can be for different effects. High intensity may result in tissue ablation and low intensity may result in a stimulating effect, creating a metabolic increase or stimulating tissue growth.

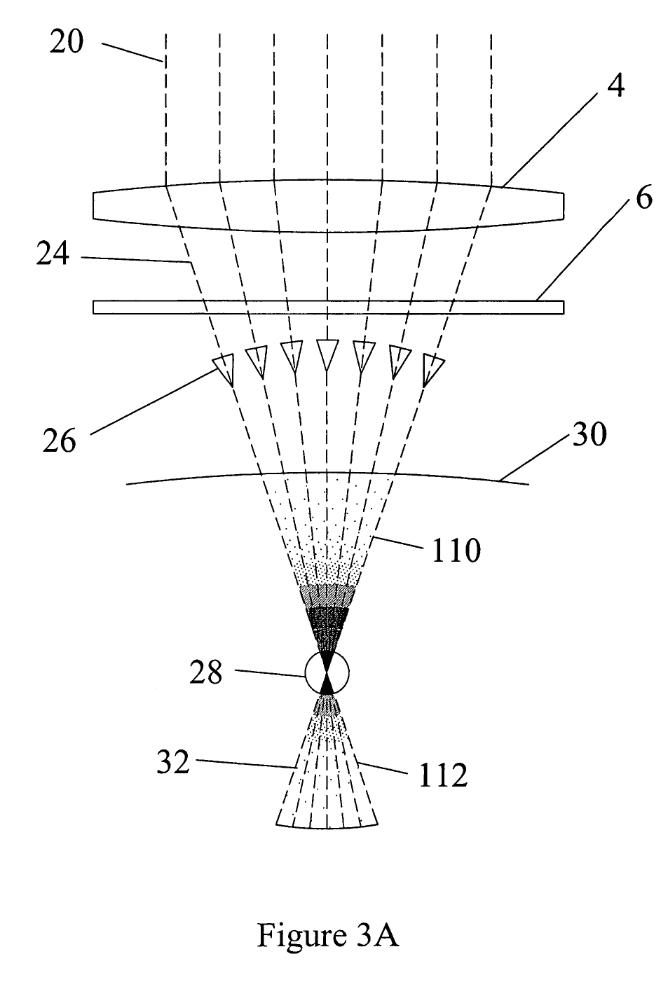

FIG. 3 and FIG. 3A are conceptual drawings of the device showing energy 20 being emitted from electromagnetic energy generator 2. Energy 20 is denoted by dashed lines, while arrows 26 denote the direction of the energy. The energy passes through focusing lens 4 and is focused by focusing lens 4, with focused energy 24 being denoted by dashed lines. The energy passes through optional lens protector 6 through surface of the subject 30 to target 28. Target 28 is the focal point of lens 4. The energy then passes through the target with dashed lines denoting post-target energy 32.

FIG. 3A has shading to denote the amount of energy per volume, with the darker areas correlating to a higher amount of energy per volume. Normally, the target 28 would be tissue that needed to be modified or destroyed. Most targets are below entry surface 30. Normally this surface 30 will be the skin or epidermal tissue layer, but in some cases lower layers of tissue and/or bone are removed before treatment with this energy generating device. So in general, the surface 30 would indicate the first area of energy contact that has significant energy absorption properties. Pre-target sub-surface subject area 110 ranges from surface 30 to target 28. Near surface 30 there is a minimal amount of energy per volume which is denoted by minimal shading in area 110. As the energy penetrates the subject, the focusing effect of the lens creates a continually greater energy per volume ratio, denoted by a darkening of shading as the energy approaches target 28. When the energy reaches the target, there is a maximal energy per volume ratio, denoted by maximal shading. Post-target sub surface subject area 112 includes areas beyond the target. As energy travels farther away from target 28, there is a decreasing energy per volume ratio, denoted by a decreasing amount of shading in area 112. As the energy enters the subject at surface 30, some energy is absorbed by every layer of tissue. This means that that every successive layer of tissue father away from surface 30 will have less total energy. While the focusing effect of the lens 4 is far greater than the total energy loss per layer of tissue and the energy per volume ratio is largely unaffected by the total energy loss in area 110, it does mean that area 110 will have a greater energy per volume ratio than area 112. This greater energy per volume ratio is denoted by area 110 having overall darker shading than area 112.

The size and shape of lens 4 is approximate. The size and shape of the lens determines the focal point of the lens. A wider lens will result in the energy initially being spread out wider before being focused to target 28. This would result in less energy being absorbed by each cell of the non-target tissue. Different electromagnetic wavelengths often require lenses of different materials. Lenses of appropriate size, shape and material may be selected as needed.

This device can be configured to use different electromagnetic energy sources which will determine the effect of this device on the target tissue.

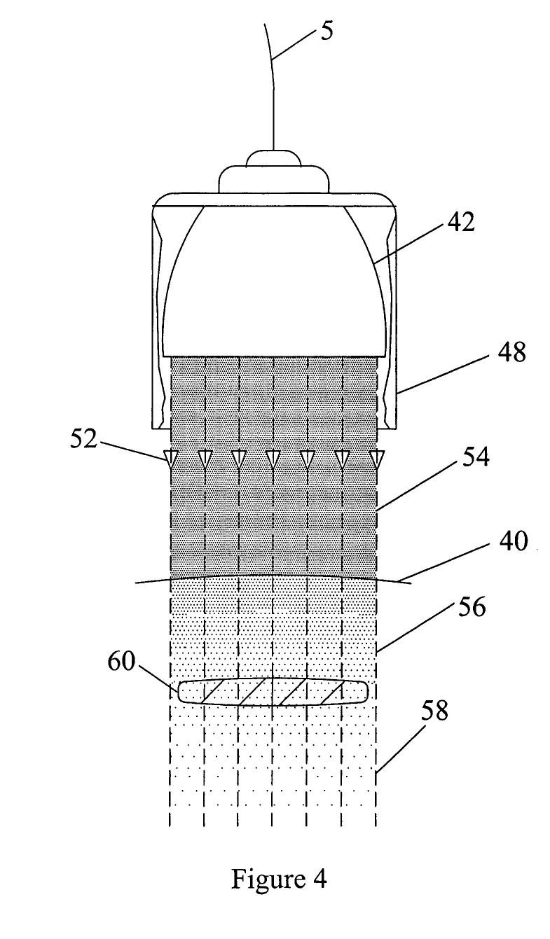

FIG. 4 is a general diagram of prior art inventions used for tissue treatment. Prior inventions used for sub-dermal tissue alteration including tissue ablation and metabolic increase use a linear method of energy generation. Energy generator 42 enclosed in protective case 48 produces energy 54 in the direction of arrows 52. Communication link 5 is attached to energy generator 42. The energy reaches the subject tissue at surface 40, enters pre-target sub-surface area 56, passes through target 60, and continues through post-target sub-surface area 58. The total energy per tissue layer and energy to volume ratio are at maximum at the surface. Once the energy enters the subject, both the total energy per tissue layer and energy to volume ratio continually decrease, the farther away from the surface that the energy goes. The decrease in energy to volume ratio is denoted by a continual decrease in shading in pre-target sub-surface subject area 56, target 60, and post-target sub-surface subject area 58. The significant difference between FIG. 4 and FIG. 3A is that in FIG. 4, the maximal energy to volume ratio is at surface 40, while in FIG. 3A the maximal energy to volume ratio is at target 28. The energy to volume ratio is the main determinate in how much energy is absorbed and therefore the effect on the subject matter. Prior inventions (with few exceptions) had maximal effect near the subject's surface matter and less of an effect on the target, while the current invention has a maximum effect on the target while minimizing the effect on surrounding subject matter. This means current invention will have minimal side effects when compared to prior inventions.

In a hypothetical example using energy wavelengths that result in heating the target to the point of tissue ablation, the current invention could heat a target of zero to one millimeters from the focal point (target) twenty degrees Celsius while the tissue layer one to two millimeters from the focal point would be only be heated approximately three degrees Celsius, and the tissue layers two or more millimeters away from the focal point would be heated less than two degrees Celsius. A twenty degree Celsius increase in temperature will kill most cells, while a three degree Celsius increase in temperature is in the safe range of a normal fever and is likely to leave healthy cells unharmed. This effectively means that only the target tissue would be heated to the point of destruction, while the surrounding tissue would have minimal or no side effects. Although some inventions have attempted to solve this problem through other means besides with the use of a lens, generally prior inventions destroy most of the intervening tissue before the target and much of the tissue after the target.

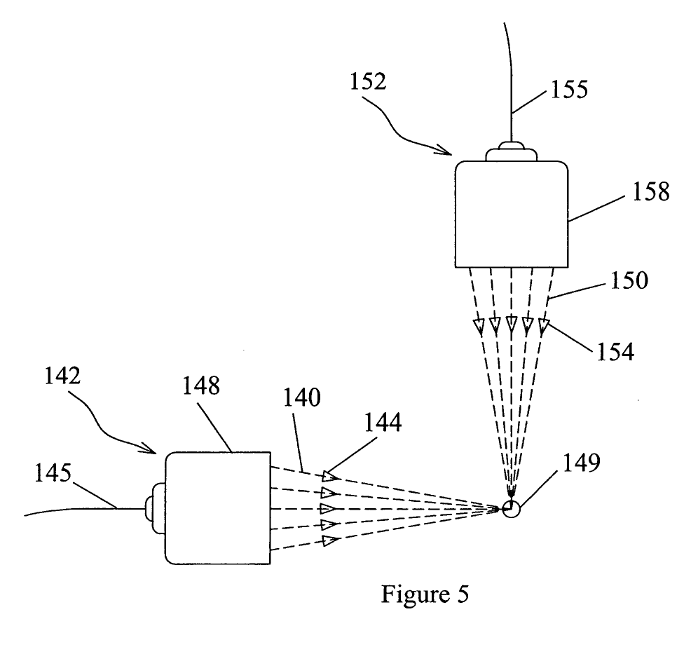

FIG. 5 shows the use of multiple devices for use on one target 149. Both the first energy generating device 142 and the second energy generating device 152 are of the specifications in FIG. 1 and FIG. 2, and include the energy generator, lens and lens protector (not visible) inside their respective protective cases 148 and 158 and have respective communication links 145 and 155. The energy generated 140 and 150 travels in the direction of arrows 144 and 154 converging on the target 149, enabling a greater concentration of energy on target 149 and reduces the energy to volume ratio on non-target tissue. If desired three or more devices could similarly be used.

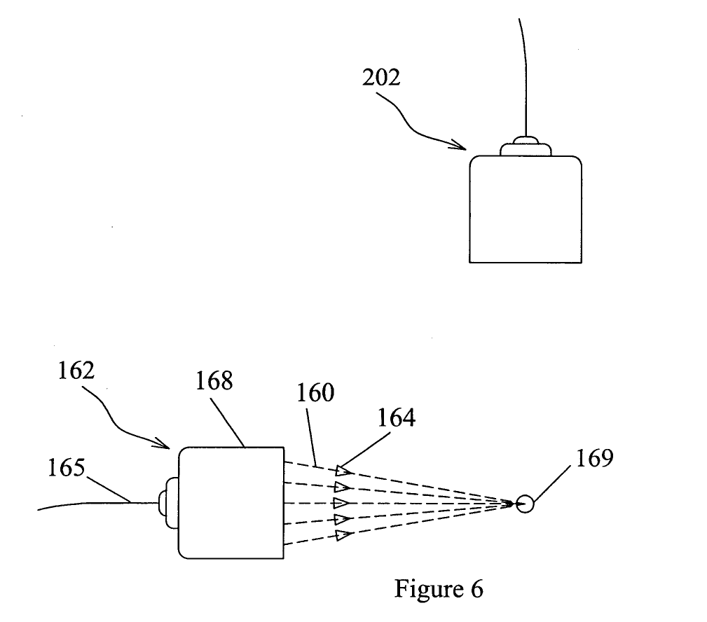

FIG. 6 is a diagram of an electromagnetic generating device 162 with targeting device 202. Electromagnetic generating device 162 is of the specification in FIG. 1 and FIG. 2, and includes an energy generator, lens and lens protector (not shown) inside the protective case 168 with communication link 165. Energy generated 160 travels in direction shown by arrows 164 toward target 169.

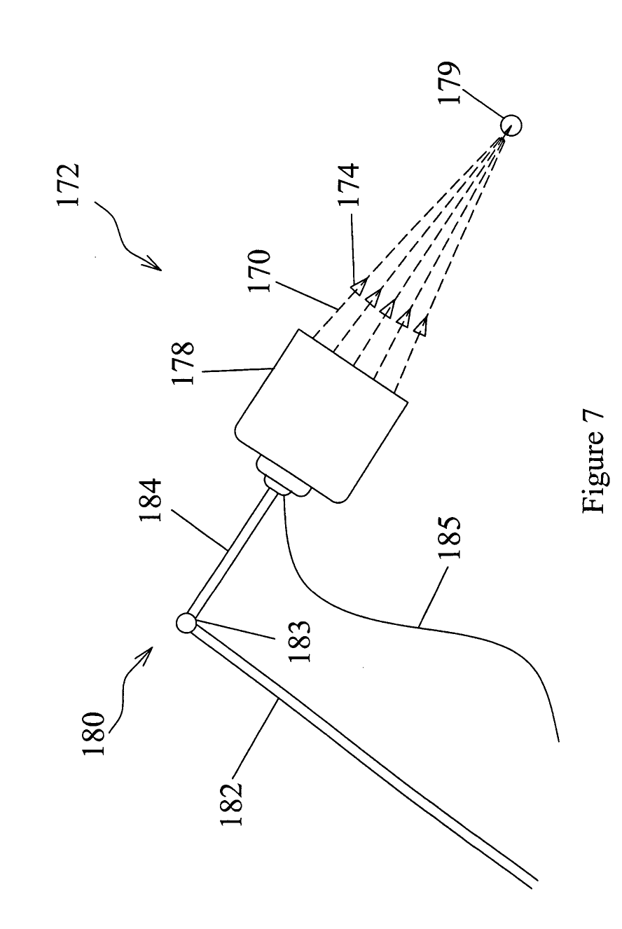

FIG. 7 is a diagram of an electromagnetic generating device 172 attached to movable support 180. Electromagnetic generating device 172 is of the specification in FIG. 1 and FIG. 2, and includes an energy generator, lens and lens protector (not shown) inside the protective case 178 with communication link 185. Energy generated 170 travels in direction shown by arrows 174 toward target 179. Mechanical support 180 is comprised of first control support 184, rotatable connector 183 and second control support 182. First shaft 184 is connected to electromagnetic generating device 172 on one end and joint 182 on the other end. Rotatable connector 183 connects first shaft 184 to second shaft 182. Mechanical support 180 may be used to accurately position energy generating device 172 for precise positioning of electromagnetic energy for maximum effect and minimal damage to surrounding tissue.

Operation of Device Described in FIG. 1 and FIG. 2:

1) Calculate exact position of the target
1a) FIG. 6 shows a targeting device 202 that when used with control box 3 can calculate the exact position of the target.
2) Position the optical center of the lens at a predetermined distance from the target so that the focal point of the lens is at the same three-dimensional point as the target.
2a) FIG. 7 shows joint 182 that can be used to position the device
3) Turn on the energy generator until the target has reached the desired temperature.
4) Turn off energy generator.
5) For large targets, repetition of the treatment will be required to cover the entire area of the target.
6) Control box 3 in FIG. 1 can be used to control the turning on and off the energy generator and can control movement of joint 182. Control box 3 can receive also receive input from targeting device 202 in FIG. 6.
   The present invention describes several embodiments. It will be apparent to a person skilled in the art that various modifications and combinations of the described components of the invention can be made without departing from the scope of this invention.

1) A device capable of 3-dimensional focusing for use in subsurface medical application comprising:
a first means capable of generating electromagnetic energy; and
a second means connected to the first means using a lens of a predetermined size and shape suitable for transmitting and focusing electromagnetic energy;
whereby said device can generate and focus electromagnetic energy at a subsurface target a predetermined distance from said device.

2) A device according to claim 1 also comprising:
a third means connected to the first means capable of moving the first means.

3) A device according to claim 2 also comprising:
a fourth means capable of determining 3-dimensional differences in the composition of body tissue;
a fifth means capable of transmitting information to and/or from the first, second, third and fourth means; and
a sixth means capable of displaying information from the first, second, third, fourth and/or fifth means.

4) A device according to claim 3 wherein a plurality of means of generating electromagnetic energy are used to focus the energy generated toward an approximate singular 3-dimensional point.

5) A device according to claim 3 wherein the energy generated by the first means is capable of heating matter.

6) A device according to claim 3 wherein the energy generated by the first means is capable of tissue ablation.

7) A device according to claim 1 wherein the electromagnetic energy generated is of a wavelength greater than 7×10⁻⁷ (m).

8) A device according to claim 1 wherein the electromagnetic energy generated is of a wavelength less than 1×10⁻⁸ (m).

9) A medical device comprising:
a first means capable of generating electromagnetic energy;
a second means connected to the first means capable of moving the first means;
a third means capable of determining 3-dimensional differences in the composition of body tissue; and
a fourth means capable of transmitting information to and/or from the first, second and third means.

10) A device according to claim 9 wherein the energy generated from the first means is focused by a fifth means comprising of a lens.

11) A device according to claim 10 wherein a plurality of means of generating electromagnetic energy are used to focus the energy generated toward an approximate singular 3-dimensional point.

12) A device according to claim 10 wherein the energy generated by the first means is capable of heating matter.

13) A device according to claim 10 wherein the energy generated by the first means is capable of tissue ablation.

14) A device according to claim 10 wherein the electromagnetic energy generated is of a wavelength greater than 7×10⁻⁷ (m)

15) A device according to claim 10 wherein the electromagnetic energy generated is of a wavelength less than 1×10⁻⁸ (m).

16) A device according to claim 10 wherein there is a sixth means capable of displaying information from the first, second, third, and/or fourth means.

17) A device comprising:
a first means capable of generating electromagnetic energy;
a second means connected to the first means using a lens of a predetermined size and shape suitable for transmitting and focusing electromagnetic energy;
a third means connected to the first means capable of moving the first means; and
a fourth means capable of transmitting information to and/or from the first, second and third means.
whereby said device can generate and focus electromagnetic energy at a target a predetermined distance from said device.

18) A device according to claim 17 wherein the electromagnetic energy generated is of a wavelength greater than 7×10⁻⁷ (m).

18) A device according to claim 17 wherein the electromagnetic energy generated is of a wavelength less than 1×10⁻⁸ (m).

19) A device according to claim 17 also comprising:
a fifth means capable of determining 3-dimensional differences in the composition of body tissue.

20) A device according to claim 17 also comprising:
a sixth means capable of displaying information from the first, second, third, forth, and/or fifth means.
